(19) United States
(12) Patent Application Publication
(10) Pub. No.: US 2014/0294742 A1
(43) Pub. Date: Oct. 2, 2014

(54) ANTI-PLAQUE DENTAL COMPOSITIONS

(71) Applicant: UL TRADENT PRODUCTS, INC., South Jordon, UT (US)

(72) Inventors: Dan E. Fischer, Sandy, UT (US); Peter M. Allred, Bluffdale, UT (US)

(21) Appl. No.: 14/350,857

(22) PCT Filed: Sep. 11, 2012
(86) PCT No.: PCT/US2012/054670

§ 371 Date: May 2, 2014

Related U.S. Application Data
(60) Provisional application No. 61/545,510, filed on Oct. 10, 2011.

(51) Int. Cl. A61K 8/22; A61K 8/21; A61K 8/34; B05B 11/00; A61K 8/31; A61Q 11/00; B65D 83/14; A61C 19/06; A61K 8/19

(52) U.S. Cl. CPC A61K 8/22; A61C 19/063; A61K 8/21; A61K 8/345; A61K 8/19; A61K 8/31; A61Q 11/00; B65D 83/752; B05B 11/3042
USPC 424/52; 424/53; 222/394; 222/383.1

(57) ABSTRACT

Anti-plaque dental treatment compositions are useful in treating and/or preventing formation of dental plaque and biofilm. The anti-plaque compositions are configured for daily application to a patient's teeth in order to inhibit dental plaque formation and generally include a peroxide-based anti-bacterial compound, xylitol in an amount so as to provide a bacteriostatic effect, and an aqueous gel carrier comprised of water and at least one thickening agent into which the anti-bacterial compound and xylitol are dispersed.

### BACKGROUND OF THE INVENTION

1. The Field of the Invention

The present invention is in the field of dental treatment compositions, including compositions for reducing dental plaque.

2. The Relevant Technology

Geriatric and incapacitated patients often suffer from serious dental infections resulting from dental plaque due to a general inability to engage in proper tooth brushing. Dental plaque is a biofilm, often pale yellow, that develops naturally on the teeth. Like any biofilm, dental plaque is formed by colonizing bacteria trying to attach themselves to a smooth surface (i.e., of a tooth).

The oral cavity contains the only known anatomical aspect of the human body that does not have a regulated system of shedding surfaces. This allows many species of microorganisms to adhere to the surface of teeth for prolonged time periods. Dental plaque is composed of about a thousand different types of bacteria that take part in the complex ecosystems of the mouth. The natural, non-frequent regulation of tooth shedding plays a substantial role in making dental biofilm the most diverse biofilm in the human body despite the relatively small size of the teeth. Bacteria that make up dental plaque have the ability to change their environment through a series of biotic relationships.

Dental plaque is initially soft enough so that it can be removed using a finger nail. Within 48 hours, however, it starts to harden, and in about 10 days plaque becomes dental calculus (tartar), which is rock-hard and difficult to remove. Failure to promptly and frequently remove plaque by daily brushing can cause long-term buildup of calculus, which can cause chronic irritation and inflammation, such as gingivitis and chronic periodontitis. Dental plaque can also give rise to dental caries (tooth decay)—the localized destruction of the tissues of the tooth by acid produced from the bacterial degradation of fermentable sugars.

Peroxides have been used as oral disinfectants and can also whiten teeth. While dental bleaching compositions typically contain sufficient peroxide to have an antibacterial effect, they can also cause tissue irritation and sensitivity to hot and cold temperatures, especially if used every day.

### BRIEF SUMMARY

Disclosed herein are anti-plaque dental treatment compositions for use in treating and/or preventing formation of dental plaque and biofilm on a patient's teeth, such as in geriatric and incapacitated patients. The anti-plaque compositions are configured for daily application to a patient's teeth in order to inhibit dental plaque formation and generally include a peroxide-based anti-bacterial compound, xylitol in an amount so as to provide a bacteriostatic effect, and an aqueous gel carrier comprised of water and at least one thickening agent into which the anti-bacterial compound and xylitol are dispersed.

According to one embodiment, the peroxide-based anti-bacterial compound is included in an amount so as to not cause significant soft tissue irritation and/or sensitivity to hot and/or cold temperature. Because of the synergistic interplay between the anti-bacterial action of the anti-bacterial compound and the bacteriostatic effect of the xylitol, it may be unnecessary to include an amount of peroxide-based anti-bacterial compound that will kill substantially all bacteria in a person's mount by itself. While dental bleaching compositions include sufficiently high amounts of peroxide such that they may also provide an anti-bacterial effect, they are typically too harsh to be used on a daily basis over prolonged periods of time (e.g., months or years during patient convalescence). Accordingly, it may be desirable to include sufficiently low quantities of peroxide-based anti-bacterial compound that may not be efficacious for bleaching teeth using conventional tooth bleaching methods (although when used over a prolonged time period of months or years, even low amounts of peroxide may exhibit some degree of bleaching).

In addition to the peroxide-based anti-bacterial compound, xylitol is included in sufficiently high amounts so as to not only provide a sweetening or flavoring effect, but also a bacteriostatic effect. The inventors have determined that, when used in combination with the peroxide-based anti-bacterial compound, xylitol exhibits a bacteriostatic effect when included in an amount of at least about 15% by weight, preferably at least about 20% by weight, and more preferably at least about 25% by weight. Xylitol provides a bacteriocidal effect because it is a sugar alcohol that mimics sugars such as glucose, xylose, fructose, sucrose and the like but cannot be metabolized by bacteria to provide energy and support life. As a result, as bacteria in the mouth consume xylitol instead of sugar, they progressively starve to death and die. In combination with the peroxide-based anti-bacterial compound, daily treatment with xylitol (e.g., 30-60 minute treatments once or twice daily) kills bacteria and removes and/or prevents formation of dental plaque and biofilm. To the extent a patient has substantial buildup of calculus or tartar, it may be useful to include anti-tartar compounds in the treatment compositions and/or physically remove such buildup using prophylaxis paste.

Because of the inclusion of relatively high quantities of xylitol, it is typically desirable to include sufficient water to substantially solvate the xylitol, which is a solid, water-soluble crystalline powder at room temperature. The amount of water to be included may depend on the amount of xylitol and other water soluble or water-gellable components within the composition (e.g., thickening agents, stabilizing agents, tooth remineralizing compounds, desensitizing agents, neutralizing bases, and the like).

Thickening agents can provide the compositions with the consistency of a gel, including non-foaming gels and also foamable gels that include foaming agents (e.g., isopentane) and are converted into a composition having a consistency similar to that of shaving cream or whipping cream.

According to one embodiment, the compositions will be substantially free of polyols other than xylitol, particularly polyols that do not themselves provide a bacteriostatic effect.

The compositions may include other adjuvents as desired to yield compositions having predetermined properties. These include, but are not limited to, comprising anti-tartar agents, dental anesthetics, gingival soothing agents, desensitizing agents, stabilizing agents, remineralizing agents, mouth freshening agents, and anti-oxidants.

In the case of foamable anti-plaque compositions, it may be advantageous to store and dispense the compositions using a pressurized container, such as a can used to dispense foamable toothpastes. Pumps may also be used to dispense foamable anti-plaque compositions. Non-foaming compositions may be dispensed using dispensing means known in the art for dispensing dental treatment compositions, including syringes, bulbs, tubes, bottles, pressurized cans, and pumps.

The anti-plaque dental treatment compositions are advantageously applied to a patient's teeth using a moisture-resistant barrier layer that holds the compositions over a patient's tooth surfaces and helps prevent premature dilution of the compositions by saliva and/or substantial spread of the compositions throughout the patient's mouth during treatment. Example barrier layers include, but are not limited to, thin, flexible strips such as those used to apply dental bleaching compositions, thin and flexible customized dental treatment trays, non-customized delivery trays, and sports mouth guards.

These and other advantages and features of the present invention will become more fully apparent from the following description and appended claims, or may be learned by the practice of the invention as set forth hereinafter.

### DETAILED DESCRIPTION OF THE PREFERRED EMBODIMENTS

Preferred embodiments of the disclosure include improved anti-plaque dental treatment compositions used to treat plaque and biofilm on a patient's teeth, such as geriatric and incapacitated patients. The anti-plaque compositions are designed for daily application to a patient's teeth in order to inhibit dental plaque formation, kill bacteria, and remove biofilm. They generally include a peroxide-based anti-bacterial compound, xylitol in an amount so as to provide a bacteriostatic effect, and an aqueous gel carrier comprised of water and at least one thickening agent into which the anti-bacterial compound and xylitol are dispersed. The compositions can be non-foaming or foamable.

The term “barrier layer”, as used herein, refers to one or more layers of a material that protects the treatment composition from ambient moisture and saliva found within a person's mouth. The barrier layer may be a thin flexible strip that can be folded over a person's teeth to form a tray-like structure. Alternatively, the barrier layer may be a non-customized dental tray that is devoid of indentations or other structures corresponding to a patient's unique dentition. The barrier can alternatively be a custom-fitted dental tray that has been customized using the patient's teeth or a stone model of the patient's teeth as a template.

The term “sticky, viscous gel” shall refer to treatment compositions that have been formulated or processed so that they do not readily flow by the force of gravity but are viscous so that they can be expressed from a syringe orifice or other dispensing means known in the art.

Anti-plaque dental treatment compositions according to the disclosure may have any desired consistency, including but not limited to a gel (e.g., a sticky, viscous gel) or a foamed composition having the consistency of shaving cream or whipping cream. In general, treatment compositions will include at least one peroxide-based anti-bacterial agent that is effective in killing at least some plaque- and biofilm-forming bacteria on a patient's tooth surfaces together with a sufficient quantity of xylitol so as to provide a bacteriostatic effect when applied to a patient's teeth on a to daily basis in combination with the peroxide-based anti-bacterial agent. The peroxide-based anti-bacterial agent and xylitol are preferably dispersed in an aqueous-based carrier comprised of water and at least one thickening agent.

Examples of peroxide-based anti-bacterial agents include, but are not limited to, one or more of aqueous hydrogen peroxide, carbamide peroxide, metal perborates (e.g., sodium perborate), metal percarbonates (e.g., sodium percarbonate), metal peroxides (e.g., calcium peroxide), metal chlorites and hypochlorites, peroxy acids (e.g., peroxyacetic acid), and peroxy acid salts. The peroxide-based anti-bacterial agent is preferably included in an amount so as to not cause significant soft tissue irritation and/or sensitivity to hot and/or cold temperature. Because of the synergistic interplay between the anti-bacterial action of the anti-bacterial compound and the bacteriostatic effect of the xylitol, it may be unnecessary to include an amount of peroxide-based anti-bacterial compound that will kill substantially all bacteria in a person's mount by itself. While dental bleaching compositions include sufficiently high amounts of peroxide such that they may also provide an anti-bacterial effect, they are typically too harsh to be used on a daily basis over prolonged periods of time (e.g., months or years during patient convalescence). Accordingly, it may be desirable to include sufficiently low quantities of peroxide-based anti-bacterial compound that may not be efficacious for bleaching teeth using conventional tooth bleaching methods (although when used over a prolonged time period of months or years, even low amounts of peroxide may exhibit some degree of bleaching).

According to one embodiment, the peroxide-based anti-bacterial agent is included in an amount in a range of about 3% to about 15%, preferably in an amount in a range of about 4% to about 10%, and more preferably in an amount in a range of about 5% to about 7%.

According to one embodiment, xylitol is included in sufficiently high amounts so as to provide more than a mere sweetening or flavoring effect but also a bacteriostatic effect. The inventors have determined that, when used in combination with the peroxide-based anti-bacterial compound, xylitol exhibits a bacteriostatic effect when included in an amount of at least about 15% by weight, preferably at least about 20% by weight (e.g., about 20% to about 50% by weight), and more preferably at least about 25% by weight (e.g., about 25% to about 40% by weight). Xylitol provides a bacteriocidal effect because it is a sugar alcohol that mimics sugars such as glucose, xylose, fructose, sucrose and the like but cannot be metabolized by bacteria to provide energy and support life. As a result, as bacteria in the mouth consume xylitol instead of sugar, they progressively starve to death and die. In combination with the peroxide-based anti-bacterial compound, daily treatment with xylitol (e.g., 30-60 minute treatments once or twice daily) kills bacteria and removes and/or prevents formation of dental plaque and biofilm. To the extent a patient has substantial buildup of calculus or tartar, it may be useful to include anti-tartar compounds in the treatment compositions and/or physically remove such buildup using prophylaxis paste.

Because of the inclusion of relatively high quantities of xylitol, it is typically desirable to include sufficient water to substantially solvate the xylitol, which is a solid, water-soluble crystalline powder at room temperature. The amount of water to be included may depend on the amount of xylitol and other water soluble or water-gellable components within the composition (e.g., thickening agents, stabilizing agents, tooth remineralizing compounds, desensitizing agents, neutralizing bases, and the like). According to one embodiment, water is included in an amount in a range of about 20% to about 75% by weight of the dental treatment composition, preferably in a range of about 25% to about 70% by weight, and more preferably in a range of about 30% to about 60% by weight of the dental treatment composition.

Thickening agents can provide the compositions with the consistency of a gel, including non-foaming gels and also foamable gels that include foaming agents and are converted into a composition having a consistency similar to that of shaving cream or whipping cream. Examples of suitable thickening agents include, but are not limited to polyvinyl pyrrolidone (PVP), carboxypolymethylene, polyethylene oxide, polyacrylic acid, copolymer of polyacrylic acid, polyacrylate, polyacrylamide, copolymer of polyacrylic acid and polyacrylamide, PVP-vinyl acetate copolymer, poly(2-ethyl-2-oxazoline), carboxymethylcellulose, carboxypropylcellulose, polysaccharide gums (e.g., carrageenan), proteins, fumed silica, and fumed alumina.

An example of a commercially available thickening agent is Carbopol 974, which is a polyacrylic acid cross-linked with a polyalkenyl ethers and is also known as carboxypolymethylene. Another example is Carbopol Ultrez 20, which is a hydrophobically modified cross-linked acrylate copolymer. Yet another example is Carbopol ETD 2020, a cross-linked polyacrylic acid copolymer. Other examples are Pluronic F68 and Pluronic F127, which are different grades of polyoxyethylene-polyoxypropylene block copolymers. In the case of foamable compositions, Pluronic F68 and Pluronic F127 can act as foam stabilizers. Polyox is an example of a polyethylene oxide made by Union Carbide. Pemulen is an example of a polyacrylic acid copolymer. Examples of PVP include Kollidon 30 made by BASF and having a molecular weight of 50,000, Kollidon VA 60 having a molecular weight of 60,000, and Kollidon 90 F having a molecular weight of 1.3 million.

According to one embodiment, the thickening agent is included in an amount in a range of about 2% to about 50% by weight of the dental treatment composition, preferably in a range of about 4% to about 30% by weight, and more preferably in a range of about 6% to about 20% by weight of the dental treatment composition.

According to one embodiment, the compositions will be substantially free of polyols other than xylitol, particularly polyols that do not themselves provide a bacteriostatic effect. Other polyols that may be included in amounts less than about 20% by weight, preferably less than about 10% by weight, more preferably less than about 8% by weight, include glycerin, mannitol, other sugar alcohols, propylene glycol, 1,3-propanediol, polyethylene glycol, and polypropylene glycol.

The compositions may include other adjuvents as desired to yield compositions having predetermined properties. These include, but are not limited to, comprising anti-tartar agents (e.g., pyrophosphate salts), dental anesthetics (e.g., benzocaine, lidocain, and the like), gingival soothing agents (e.g., aloe vera, mild potassium nitrate, isotonic solution-forming salts), stabilizing agents (e.g., EDTA, salts of EDTA, citric acid and its salts, phosphoric acid and its salts, phenolphosphonic acid and its salts, gluconic acid and its salts, alkali metal pyrophosphates, alkali metal polyphosphates, and alkyl sulfates), desensitizing agents (e.g., potassium nitrate, other potassium salts, citric acid, citrates, and sodium fluoride), remineralizing agents (e.g., sodium fluoride, stannous fluoride, sodium monofluorophosphate, and other fluoride salts), mouth freshening agents (e.g., camphor and oil of wintergreen), preservatives (e.g., chlorhexidine, triclosan, sodium benzoate, parabens, tetracycline, phenols, and cetyl pyridinium chloride), and anti-oxidants (e.g., vitamin A, vitamin C, vitamin E, other vitamins, and carotene), neutralizing agents (e.g., sodium hydroxide and triethanolamine), colorants, flavorants, and sweeteners.

According to one embodiment, the anti-plaque dental treatment compositions include sodium fluoride or other fluoride salt in an amount so as to provide a remineralizing and/or anticariogenic effect. This helps improve overall dental health in combination with removing or inhibiting formation of dental plaque. The fluoride salt may be included in an amount in a range of about 0.01% to about 2%, preferably about 0.025% to about 1.5%, more preferably about 0.05% to about 1% by weight of the composition. For over-the-counter formulations, the amount of fluoride salt may be in a range of about 0.01% to about 0.5% (e.g., 0.254%). For prescription products, the amount of sodium fluoride may be in a range of about 0.05% to about 2% by weight (e.g., 0.5% or 1.1%). Sodium fluoride is preferred, but other fluoride salts may be used in amounts so as to provide similar stoichiometric amounts of fluoride ion. When fluoride is included in prescription quantities, it may also provide a desensitizing effect that offsets sensitivity that may be caused by the peroxide-based anti-bacterial agent.

According to one embodiment, the anti-plaque dental treatment compositions include potassium nitrate or other desensitizing agent in an amount so as to provide a desensitizing effect that offsets sensitivity that may be caused by the peroxide-based anti-bacterial agent. This helps improve overall dental health by promoting patient compliance. The desensitizing agent may be included in an amount in a range of about 0.25% to about 5% by weight, preferably about 0.5% to about 3% by weight of the dental composition. According to one embodiment, potassium nitrate and fluoride are not included together in the same composition. In another embodiment, both can be included.

In the case of foamable anti-plaque compositions, it may be advantageous to store and dispense the compositions using a pressurized container, such as a can used to dispense foamable toothpastes. Pumps may also be used to dispense foamable anti-plaque compositions with or without blowing agents such as isopentane. Pluronic polymers can assist in stabilizing foams formed from rapid movement and/or shear from a pump where an expanding gas (e.g., isopentane) is not used. Non-foaming compositions may be dispensed using dispensing means known in the art for dispensing dental treatment compositions, including syringes, bulbs, tubes, bottles, pressurized cans, and pumps.

In the case of foamable anti-plaque dental treatment composition, it may be advantageous to include at least one foaming agent in an amount so as to provide a foaming effect when the composition is dispensed from a pressurized container or using a pump. Examples of foaming agents including, but are not limited to, isopentane and pentane. The foaming agent may be included in an amount up to about 5% by weight, preferably up to about 3% by weight, and preferably up to about 2% by weight of the treatment composition.

According to one embodiment, anti-plaque dental treatment compositions are provided that are substantially void of abrasives (e.g., have an insufficient amount of solid particulates so as to not significantly abrade a patient's teeth when applied thereto) (e.g., less than about 5%, preferably less than about 3%, more preferably less than about 1%, and most preferably essentially free of abrasives).

The anti-plaque dental treatment compositions are advantageously applied to a patient's teeth using a moisture-resistant barrier layer that holds the compositions over a patient's tooth surfaces and helps prevent premature dilution of the compositions by saliva and/or substantial spread of the compositions throughout the patient's mouth during treatment. Example barrier layers include, but are not limited to, thin, flexible strips such as those used to apply dental bleaching compositions, thin and flexible customized dental treatment trays, non-customized delivery trays, and sports mouth guards.

Examples of liquid or gel solvents, carriers or vehicles include, but are not limited to, water, alcohols (e.g., ethyl alcohol), and polyols (e.g., glycerin, sorbitol, mannitol, other sugar alcohols, propylene glycol, 1,3-propanediol, polyethylene glycol, polyethylene oxide, and polypropylene glycol).

In the case of compositions that are substantially solid or a stiff putty, the concentration of solvent, carrier or vehicle will typically be attenuated compared to treatment gels. Where it is desired to form a treatment gel that is later converted into a putty or solid composition, it may be advantageous to include one or more volatile solvents that can be removed by evaporation (e.g., water, alcohols, acetone, and other organic solvents). Because of the affinity of hydrophilic polymers for water, even treatment compositions that appear to be solid may include a significant amount of bound water (e.g., up to about 10% or more by weight of the treatment composition). In the case where the treatment composition has the consistency of a highly viscous or stiff putty, the composition will generally include a solvent, carrier or vehicle that acts as a plasticizer or softening agent.

Barrier layers used in applying the anti-plaque dental treatment compositions to a patient's teeth are preferably moisture-resistant in order to protect the treatment composition from ambient moisture found in a person's mouth. According to one embodiment, the barrier layer comprises a thin, flexible membrane formed from a moisture-resistant polymer material. Thin, flexible barrier layers preferably have a thickness in a range of about 0.0001 inch to about 0.012 inch, more preferably in a range of about 0.001 inch to about 0.01 inch. The barrier layers may be capable of forming a dental tray or tray-like device absent an endoskeleton, or they may be shapeless requiring an endoskeleton. Alternatively, thicker dental trays, both custom and non-custom, can be used, including athletic mouth guards.

Examples of materials that can be used to form the barrier layer include, but are not limited to, polyolefins, wax (e.g., paraffin wax), metal foil, paraffin, ethylene-vinyl acetate copolymer (EVA), ethylene-vinyl alcohol copolymer (EVAL), polycaprolactone (PCL), polyvinyl chloride (PVC), polyesters, polycarbonates, polyamides, polyurethanes, polyesteramides, and blends thereof. The barrier layer may comprise a blend and/or multiple layers comprising two or more of the foregoing materials. An example of a barrier layer comprising a blend of polyolefins and paraffin wax is Parafilm®. Plasticizers, flow additives, and fillers known in the art can be used as desired to modify the properties of any of the foregoing polymers used to form the barrier layer.

The anti-plaque dental treatment compositions can be applied for as little as a few minutes or as long as several hours. By way of example, not limitation, a typical treatment session of fast duration may last from about 10 to about 30 minutes. A treatment session of intermediate duration may last from about 30 minutes to about 2 hours. A treatment session of long duration, including professional or overnight treatment while a person is sleeping, may last from about 2 hours to about 12 hours. In general, treatments of 30 minutes to 60 minutes once or twice daily may be adequate to provide clinically significant plaque reduction in geriatric and incapacitated patients. The anti-plaque regimen may continue for weeks, months, or even years during patient convalescence in order to eliminate or reduce potential health risks associated with dental plaque, gingival inflammation, and tooth decay.

For convenience of use, dental treatment compositions may be packaged to together with one or more barrier layers and sold as a kit. The treatment compositions may be packaged in single-used or multiple use containers or dispensing devices.

The following are several examples of anti-plaque dental treatment compositions within the scope of the invention. The exemplary formulations are given by way of example, not by limitation, in order to illustrate anti-plaque compositions that have been found to be useful in killing bacteria and removing biofilm and plaque from a patient's teeth. Unless otherwise indicated, all percentages are by weight.

Exemplary anti-plaque dental treatment compositions can be either foaming or non-foaming and can be delivered to a moisture-resistant barrier layer using a pressurized can, pump, syringe, bulb, or bottle depending on their rheology and mode of delivery. They can be applied to a patient's teeth using a variety of different barrier layers, including folding strips that can be wrapped around the teeth or pre-formed dental trays. Dental trays can be either customized or non-custom trays.

Examples 1-9 are directed to non-foaming anti-plaque compositions in the form of a gel, and Examples 10-12 are foaming anti-plaque compositions that can be delivered from a can or a pump, enabling the material to foam up similar to shaving cream or whipped cream.

### Example 1

A non-foaming, anti-plaque dental treatment composition is formed by mixing together the following components:

| | |
|---|---|
| Water | 54.70% |
| Carbamide Peroxide | 7.50% |
| EDTA Disodium Salt | 0.20% |
| Xylitol | 15.00% |
| Glycerin | 8.00% |
| Sodium Fluoride | 0.25% |
| Flavor | 0.20% |
| Potassium Nitrate | 0.50% |
| Sodium Hydroxide | 1.15% |
| Carbopol 974 | 2.50% |
| Pluronic F68 | 10.00% |
| Total | 100.00% |

The anti-plaque treatment composition has the consistency of a sticky, viscous gel that is able to assist in retaining a flexible strip, customized dental tray, or non-custom tray against a patient's teeth for a period of time of at least about 60 minutes. The carbamide peroxide kills at least a portion of plaque and biofilm forming bacteria present on a patient's teeth. The potassium nitrate provides a desensitizing effect to reduce or eliminate tissue irritation and/or sensitivity to hot or cold that may otherwise be caused by the carbamide peroxide anti-bacterial agent. The fluoride provides anticariogenic, remineralizing and desensitizing effects. The xylitol provides a bacteriostatic effect by effectively starving bacteria of nutrients when absorbed by bacteria instead of sugar.

### Example 2

A non-foaming, anti-plaque dental treatment composition is formed by mixing together the following components:

| | |
|---|---|
| Water | 54.70% |
| Carbamide Peroxide | 7.50% |
| EDTA Disodium Salt | 0.20% |
| Xylitol | 15.00% |
| Glycerin | 8.00% |
| Sodium Fluoride | 0.25% |
| Flavor | 0.20% |
| Potassium Nitrate | 0.50% |
| Sodium Hydroxide | 1.15% |
| Carbopol ETD 2020 | 2.50% |
| Pluronic F68 | 10.00% |
| Total | 100.00% |

The anti-plaque treatment composition has similar rheology, anti-plaque, and other properties as the composition of Example 1.

### Example 3

A non-foaming, anti-plaque dental treatment composition is formed by mixing together the following components:

| | |
|---|---|
| Water | 51.05% |
| Carbamide Peroxide | 7.50% |
| EDTA Disodium Salt | 0.20% |
| Xylitol | 15.00% |
| Glycerin | 8.00% |
| Sodium Fluoride | 0.25% |
| Flavor | 0.20% |
| Potassium Nitrate | 0.50% |
| Sodium Hydroxide | 2.30% |
| Carbopol ETD 2020 | 5.00% |
| Pluronic F68 | 10.00% |
| Total | 100.00% |

The anti-plaque treatment composition has similar rheology, anti-plaque, and other properties as the compositions of Examples 1 and 2.

### Example 4

A non-foaming, anti-plaque dental treatment composition is formed by mixing together the following components:

| | |
|---|---|
| Water | 54.90% |
| Carbamide Peroxide | 7.50% |
| EDTA Disodium Salt | 0.20% |
| Xylitol | 25.00% |
| Glycerin | 8.00% |
| Sodium Fluoride | 0.05% |
| Flavor | 0.20% |
| Potassium Nitrate | 0.50% |
| Sodium Hydroxide | 1.15% |
| Carbopol 974 | 2.50% |
| Total | 100.00% |

The anti-plaque treatment composition has a less sticky and viscous rheology compared to the compositions of Examples 1-3 as a result of including a lower quantity of thickening agent but has greater anti-plaque forming properties as a result of including a greater quantity of xylitol.

### Example 5

A non-foaming, anti-plaque dental treatment composition is formed by mixing together the following components:

| | |
|---|---|
| Water | 58.00% |
| Carbamide Peroxide | 7.50% |
| EDTA Disodium Salt | 0.10% |
| Xylitol | 30.00% |
| Sodium Fluoride | 0.05% |
| Flavor | 0.20% |
| Potassium Nitrate | 0.50% |
| Sodium Hydroxide | 1.15% |
| Carbopol Ultrez 20 | 2.50% |
| Total | 100.00% |

The anti-plaque treatment composition has similar rheology the composition of Example 4 and has greater anti-plaque forming properties than Examples 1-4 as a result of including an even greater quantity of xylitol.

### Example 6

A non-foaming, anti-plaque dental treatment composition is formed by mixing together the following components:

| | |
|---|---|
| Water | 45.79% |
| Carbamide Peroxide | 10.00% |
| EDTA Disodium Salt | 0.01% |
| Xylitol | 35.00% |
| Sodium Fluoride | 0.05% |
| Potassium Nitrate | 0.50% |
| Sodium Hydroxide | 1.15% |
| Carbopol ETD 2020 | 2.50% |
| Polyvinyl Pyrrolidone | 5.00% |
| Total | 100.00% |

The anti-plaque treatment composition is more sticky and viscous compared to the compositions of Examples 4 and 5 and has greater anti-plaque forming properties than Examples 1-5 as a result of including an even greater quantity of xylitol and an additional quantity of carbamide peroxides.

### Example 7

A non-foaming, anti-plaque dental treatment composition is formed by mixing together the following components:

| | |
|---|---|
| Water | 53.19% |
| Carbamide Peroxide | 7.50% |
| EDTA Disodium Salt | 0.01% |
| Xylitol | 20.00% |
| Sodium Fluoride | 1.10% |
| Flavor | 0.10% |
| Potassium Nitrate | 3.00% |
| Sodium Hydroxide | 0.10% |
| Polyvinyl Pyrrolidone | 15.00% |
| Total | 100.00% |

The anti-plaque treatment composition is more sticky and viscous compared to the compositions of Examples 4-6, has greater desensitizing ability, and has somewhat higher anti-plaque forming properties compared to Examples 1-3.

### Example 8

A non-foaming, anti-plaque dental treatment composition is formed by mixing together the following components:

| | |
|---|---|
| Water | 33.19% |
| Carbamide Peroxide | 7.50% |
| EDTA Disodium Salt | 0.01% |
| Xylitol | 20.00% |
| Sodium Fluoride | 1.10% |
| Flavor | 0.10% |
| Potassium Nitrate | 3.00% |
| Sodium Hydroxide | 0.10% |
| Poly(2-ethyl-2-oxazoline) | 35.00% |
| Total | 100.00% |

The anti-plaque treatment composition has adequate viscosity, desensitizing ability, and anti-plaque forming properties.

### Example 9

A non-foaming, anti-plaque dental treatment composition is formed by mixing together the following components:

| | |
|---|---|
| Water | 48.19% |
| Carbamide Peroxide | 7.50% |
| EDTA Disodium Salt | 0.01% |
| Xylitol | 38.00% |
| Sodium Fluoride | 1.10% |
| Flavor | 0.10% |
| Potassium Nitrate | 3.00% |
| Sodium Hydroxide | 0.10% |
| Pluronic F68 | 2.00% |

The anti-plaque treatment composition has adequate viscosity, high desensitizing ability, and high anti-plaque forming properties.

### Example 10

A non-foaming, anti-plaque dental treatment composition is formed by mixing together the following components:

| | |
|---|---|
| Water | 48.19% |
| Carbamide Peroxide | 7.50% |
| EDTA Disodium Salt | 0.01% |
| Xylitol | 38.00% |
| Sodium Fluoride | 1.10% |
| Flavor | 0.10% |
| Sodium Hydroxide | 0.10% |
| Pluronic F68 | 5.00% |

The anti-plaque treatment composition has higher viscosity and lower desensitizing ability compared to Example 9, and similar high anti-plaque forming properties.

### Example 11

A non-foaming, anti-plaque dental treatment composition is formed by mixing together the following components:

| | |
|---|---|
| Water | 48.19% |
| Carbamide Peroxide | 7.50% |
| EDTA Disodium Salt | 0.01% |
| Xylitol | 38.00% |
| Sodium Fluoride | 2% |
| Flavor | 0.10% |
| Sodium Hydroxide | 0.10% |
| Pluronic F68 | 4.10% |

The anti-plaque treatment composition has slightly lower viscosity and substantially higher remineralization and desensitizing effects compared to Example 10, and similar high anti-plaque forming properties.

### Example 12

A non-foaming, anti-plaque dental treatment composition is formed by mixing together the following components:

| | |
|---|---|
| Water | 53.19% |
| Carbamide Peroxide | 7.50% |
| EDTA Disodium Salt | 0.01% |
| Xylitol | 20.00% |
| Sodium Fluoride | 1.10% |
| Flavor | 0.10% |
| Potassium Nitrate | 3.00% |
| Sodium Hydroxide | 0.10% |
| Carrageenan | 15.00% |
| Total | 100.00% |

The anti-plaque treatment composition has adequate viscosity, desensitizing ability, and anti-plaque forming properties.

### Example 13

A foaming, anti-plaque dental treatment composition is formed by mixing together the following components:

| | |
|---|---|
| Water | 62.22% |
| Carbamide Peroxide | 5.00% |
| EDTA Disodium Salt | 0.20% |
| Xylitol | 15.00% |
| Sodium Hydroxide | 0.19% |
| Carbopol ETD 2020 | 0.40% |
| Pluronic F68 | 15.00% |
| Isopentane | 2.00% |
| Total | 100.00% |

The anti-plaque treatment composition when initially dispensed has the consistency of a sticky, viscous gel but expands into a stiff foam as a result of evaporation of isopentane. The composition has adequate antibacterial and bacteriostatic properties in order to remove dental plaque and biofilm from a patient's teeth and prevent or inhibit reformation of plaque and/or biofilm when used on a daily basis.

### Example 14

A foaming, anti-plaque dental treatment composition is formed by mixing together the following components:

| | |
|---|---|
| Water | 34.79% |
| Carbamide Peroxide | 10.00% |
| EDTA Disodium Salt | 0.01% |
| Xylitol | 35.00% |
| Sodium Fluoride | 0.05% |
| Potassium Nitrate | 0.50% |
| Sodium Hydroxide | 1.15% |
| Carbopol ETD 2020 | 2.50% |
| Polyvinyl Pyrrolidone | 5.00% |
| Pluronic F127 | 10.00% |
| Isopentane | 1.00% |
| Total | 100.00% |

The anti-plaque treatment composition when initially dispensed has the consistency of a sticky, viscous gel but expands into a stiffer foam than the composition of Example 13 as a result of including less isopentane. The composition has substantially higher antibacterial and bacteriostatic properties as a result of including greater quantities of carbamide peroxide and xylitol.

### Example 15

A foaming, anti-plaque dental treatment composition is formed by mixing together the following components:

| | |
|---|---|
| Water | 47.19% |
| Carbamide Peroxide | 7.50% |
| EDTA Disodium Salt | 0.01% |
| Xylitol | 20.00% |
| Sodium Fluoride | 1.10% |
| Flavor | 0.10% |
| Potassium Nitrate | 3.00% |
| Sodium Hydroxide | 0.10% |
| Polyvinyl Pyrrolidone | 15.00% |
| Pluronic F127 | 5.00% |
| Isopentane | 1.00% |
| Total | 100.00% |

The anti-plaque treatment composition when initially dispensed has a similar consistency as the composition of Example 15. The composition has antibacterial and bacteriostatic properties intermediate those of Examples 13 and 14.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An anti-plaque dental treatment composition configured for daily application to a patient's teeth in order to inhibit dental plaque formation, comprising:
a peroxide-based anti-bacterial compound;
xylitol included in an amount so as to provide a bacteriostatic effect; and
an aqueous gel carrier comprised of water and at least one thickening agent into which the anti-bacterial compound and xylitol are dispersed.

2. An anti-plaque dental treatment composition as in claim 1, the peroxide-based anti-bacterial compound comprising at least one member selected from the group of aqueous hydrogen peroxide, carbamide peroxide, metal perborates, sodium perborate, metal percarbonates, sodium percarbonate, metal peroxides, calcium peroxide, metal chlorites, metal hypochlorites, peroxy acids, peroxyacetic acid, and peroxy acid salts.

3. An anti-plaque dental treatment composition as in claim 1, the peroxide-based anti-bacterial compound being included in an amount so as to assist in killing bacteria and removing biofilm and plaque without causing significant tissue irritation or sensitivity to hot and cold temperatures when the dental treatment composition is used on a daily basis.

4. An anti-plaque dental treatment composition as in claim 3, the peroxide-based anti-bacterial compound being included in an amount in a range of about 3% to about 15%.

5. An anti-plaque dental treatment composition as in claim 3, the peroxide-based anti-bacterial compound being included in an amount in a range of about 4% to about 10%.

6. (canceled)

7. An anti-plaque dental treatment composition as in claim 1, the xylitol being included in an amount so as to assist in killing bacteria through starvation when the dental treatment composition is used on a daily basis.

8. An anti-plaque dental treatment composition as in claim 7, the xylitol being included in an amount of at least about 15% by weight of the dental treatment composition.

9. An anti-plaque dental treatment composition as in claim 7, the xylitol being included in an amount in a range from about 20% to about 50% by weight of the dental treatment composition.

10. (canceled)

11. An anti-plaque dental treatment composition as in claim 1, the water being included in an amount in a range of about 20% to about 75% by weight of the dental treatment composition.

12. An anti-plaque dental treatment composition as in claim 1, the water being included in an amount in a range of about 25% to about 70% by weight of the dental treatment composition.

13. (canceled)

14. An anti-plaque dental treatment composition as in claim 1, the at least one thickening agent comprising at least one of polyvinyl pyrrolidone (PVP), carboxypolymethylene, polyethylene oxide, polyacrylic acid, copolymer of polyacrylic acid, polyacrylate, polyacrylamide, copolymer of polyacrylic acid and polyacrylamide, PVP-vinyl acetate copolymer, carboxymethylcellulose, carboxypropylcellulose, polysaccharide gum, protein, or fumed silica.

15. An anti-plaque dental treatment composition as in claim 1, the at least one thickening agent being included in an amount in a range of about 2% to about 50% by weight of the dental treatment composition.

16. An anti-plaque dental treatment composition as in claim 1, the at least one thickening agent being included in an amount in a range of about 4% to about 30% by weight of the dental treatment composition.

17. (canceled)

18. An anti-plaque dental treatment composition as in claim 1, further comprising at least one fluoride salt in an amount so as to provide a remineralizing and/or anti-caries effect when the dental treatment composition is used on a daily basis.

19. An anti-plaque dental treatment composition as in claim 18, the at least one fluoride salt being included in an amount so as to provide fluoride ions in an amount in a range of about 0.01% to about 2%.

20. (canceled)

21. An anti-plaque dental treatment composition as in claim 1, further comprising potassium nitrate in an amount so as to provide a desensitizing effect that offsets sensitivity that may be caused by the peroxide-based anti-bacterial compound when the dental treatment composition is used on a daily basis.

22. An anti-plaque dental treatment composition as in claim 21, the potassium nitrate being included in an amount in a range of about 0.25% to about 5%.

23. An anti-plaque dental treatment composition as in claim 21, the potassium nitrate being included in an amount in a range of about 0.5% to about 3%.

24. An anti-plaque dental treatment composition as in claim 1, the dental treatment composition contains less than about 20% by weight of polyols other than xylitol.

25. An anti-plaque dental treatment composition as in claim 1, the dental treatment composition contains less than about 10% by weight of polyols other than xylitol.

26. (canceled)

27. An anti-plaque dental treatment composition as in claim 1, the dental treatment composition being substantially void of polyols other than the xylitol.

28. An anti-plaque dental treatment composition as in claim 1, further comprising a foaming agent in an amount so as to provide a foaming anti-plaque dental treatment composition when dispensed from a pressurized container or using a pump.

29. An anti-plaque dental treatment composition as in claim 28, the foaming agent comprising up to about 5% by weight of isopentane or pentane.

30. (canceled)

31. (canceled)

32. An anti-plaque dental treatment composition as in claim 1, the dental treatment composition being substantially void of abrasives.

33. An anti-plaque dental treatment composition as in claim 1, the dental treatment composition comprising at least one active adjuvant selected from the group comprising anti-tartar agents, dental anesthetics, gingival soothing agents, stabilizing agents, remineralizing agents, mouth freshening agents, and anti-oxidants.

34. An anti-plaque dental treatment composition as in claim 1, the dental treatment composition being non-foaming.

35. An anti-plaque treatment system, comprising:
an anti-plaque dental treatment composition according to claim 1; and
a barrier layer for use in applying the composition to a person's teeth.

36. An anti-plaque treatment system as in claim 35, the barrier layer comprising at least one of wax, metal foil, paraffin, ethylene-vinyl acetate copolymer, ethylene-vinyl alcohol copolymer, polycaprolactone, polyolefin, polyethylene, high density polyethylene, low density polyethylene, ultra-low density polyethylene, polypropylene, polytetrafluoroethylene, polyester, polycarbonate, polyurethane, polyamide, or polyesteramide.

37. An anti-plaque treatment system as in claim 35, the barrier layer comprising a non-custom dental treatment tray having sufficient flexibility as to conform to a plurality of differently sized and shaped dental arches.

38. An anti-plaque treatment system as in claim 35, the barrier layer comprising a custom-fitted dental treatment tray having indentations corresponding to a patient's unique dentition.

39. A foamable anti-plaque dental treatment composition configured for daily application to a patient's teeth in order to inhibit dental plaque formation, comprising:
a peroxide-based anti-bacterial compound;
xylitol included in an amount so as to provide a bacteriostatic effect;
an aqueous gel carrier comprised of water and at least one thickening agent into which the anti-bacterial compound and xylitol are dispersed, the aqueous gel carrier containing less than about 10% by weight of polyols other than xylitol; and
at least one of isopentane or pentane in an amount up to about 5% by weight.

40. A foamable anti-plaque dental treatment composition as in claim 39, further comprising a pressurized container in which the foamable anti-plaque dental treatment composition is stored.

41. A foamable anti-plaque dental treatment composition as in claim 39, further comprising a pump used to dispense the foamable anti-plaque dental treatment composition.

42. A method of inhibiting dental plaque formation, comprising:
providing an anti-plaque dental treatment composition configured for daily application to a patient's teeth in order to inhibit dental plaque formation, the treatment composition comprising:
a peroxide-based anti-bacterial compound;
xylitol included in an amount so as to provide a bacteriostatic effect; and
an aqueous gel carrier comprised of water and at least one thickening agent into which the anti-bacterial compound and xylitol are disperse; and
applying the anti-plaque treatment composition to a patient's teeth using a barrier layer on a daily basis in order to inhibit formation and/or facilitate removal of plaque from the patient's teeth.

43. A method of inhibiting dental plaque formation as in claim 42, wherein the anti-plaque treatment composition is applied once per day.

44. A method of inhibiting dental plaque formation as in claim 42, wherein the anti-plaque treatment composition is applied twice per day.
